SEQUENCE LISTING (1 sequence)

SEQ ID NO: 1 - DNA, 22 bp, synthetic construct
  source 1..22: mol_type = other DNA
aatgagcccttgcatctaagaa

(19) United States
(12) Patent Application Publication
(10) Pub. No.: US 2026/0325642 A1
(43) Pub. Date: Oct. 22, 2026

(54) MICROBUBBLES AND METHODS OF MAKING SAME

(71) Applicant: Drexel University, Philadelphia, PA (US)

(72) Inventors: Brian Oeffinger, Philadelphia, PA (US); Margaret Wheatley, Media, PA (US)

(21) Appl. No.: 19/110,381

(22) PCT Filed: Sep. 14, 2023
(86) PCT No.: PCT/US2023/074198

§ 371 Date: Mar. 10, 2025

Related U.S. Application Data
(60) Provisional application No. 63/406,607, filed on Sep. 14, 2022.

(51) Int. Cl. A61K 41/00; A61K 9/107; A61K 33/00; A61K 47/22

(52) U.S. Cl. CPC A61K 41/0047; A61K 9/1075; A61K 33/00; A61K 47/22

(57) ABSTRACT

The present invention provides methods for preparing acoustically-sensitive microbubbles. The method includes the steps of providing a first system comprising at least one water-soluble surfactant; providing a second system comprising at least one water-insoluble surfactant, wherein providing the second system optionally comprises the step of autoclaving the second system and optionally cooling the resulting autoclaved system; and contacting the first system with the second system at the temperature below the melting point of the second surfactant, so as to produce a mixture comprising microbubbles.

### CROSS-REFERENCE TO RELATED APPLICATION(S)

This application claims the benefit of priority under 35 U.S.C. § 119(e) to U.S. Provisional Patent Application No. 63/406,607, filed Sep. 14, 2022, the contents of which are incorporated by reference herein in their entirety.

### REFERENCE TO SEQUENCE LISTING

The Sequence Listing concurrently submitted herewith as a xml file named “046528-7126WO1(01029)_Sequence Listing.xml” created on Sep. 14, 2023 and having a size of 1.9 Kilobytes is herein incorporated by reference pursuant to 37 C.F.R. § 1.52(e)(5).

### BACKGROUND OF THE INVENTION

Currently many therapeutic treatments, like cancer treatments such as chemotherapy, are systemic, whereby the chemotherapeutic agents travel through patient's entire body killing any fast-growing cells. Chemotherapy is commonly associated with several undesired side-effects including hair loss, nausea, and weakened immune system. This lack of target specificity and off target side-effects also applies to other site-specific treatments such as gene delivery. Therefore, there is a need in the art to develop localized therapy that targets a specific site without causing the undesired side-effects. The present invention addresses this unmet need.

### SUMMARY OF THE INVENTION

In one aspect the invention provides a method for preparing microbubbles. In certain embodiments, the method for preparing microbubbles, comprises the steps of:
i. providing a first system comprising at least one water-soluble surfactant;
ii. providing a second system comprising at least one water-insoluble surfactant, wherein providing the second system optionally comprises the step of autoclaving the second system and optionally cooling the resulting autoclaved system; and
iii. contacting the first system with the second system at the temperature below the melting point of the second surfactant, so as to produce a mixture comprising microbubbles.

In certain embodiments, the first system is aqueous.

In certain embodiments, the first system comprises a micelle.

In certain embodiments, the second system comprises a cream.

In certain embodiments, the method further comprises purging the surfactant mixture of step (iii) with a purging gas.

In certain embodiments, the purging gas is a perfluorocarbon (PFC).

In certain embodiments, the method further comprises washing the mixture comprising microbubbles with DI water and separating the microbubbles from the mixture by gravity filtration.

In certain embodiments, the method further comprises freeze-drying and capping the separated microbubbles under vacuum.

In certain embodiments, the method further comprises filling the freeze-dried microbubbles with a filling gas.

In certain embodiments, the filling gas is oxygen, octafluoropropane, and sulfur hexafluoride.

In certain embodiments, the at least one water soluble surfactant is selected from the group consisting of tocopheryl polyethylene glycol succinate (TPGS), cetyltrimethylammonium Bromide (CTAB), and mixtures thereof.

In certain embodiments, the at least one water-insoluble surfactant is selected from the group consisting of sorbitan monostearate, sorbitan fatty acid esters, sorbitan monopalmitate, (+) A tolopherol acid succinate (vitamin E) and mixtures thereof.

In certain embodiments, the contacting is performed at room temperature.

In certain embodiments, at least one of the first system and the second system independently comprises an additive.

In certain embodiments, the at least one additive is selected from the group consisting of a drug, a dye, and a charged molecule for surface modification of the microbubbles.

In certain embodiments, the charged molecule alters the charge (zeta potential) of the microbubbles.

In certain embodiments, the microbubbles with altered zeta potential are further loaded with oppositely charged molecules.

In certain embodiments, the microbubbles are acoustically-sensitive.

In certain embodiments, wherein the size of the separated microbubbles is below 10 μm in diameter.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the chemical structures of sorbitan monostearate and tocopheryl polyethylene glycol succinate (TPGS).

FIG. 2 depicts a schematic showing different methods, i.e., original method (OM), micelle method (MM), and cream method (CM), for preparing the microbubbles (MBs).

FIG. 3 depicts that the presently developed method (cream method) allows TPGS and sorbitan monostearate to be combined at lower temperatures, which is better for heat sensitive drug loading. The cream method also allows chemical addition to sorbitan monostearate phase at room temperature (such as drug or cationic species). However, sorbitan monostearate formed large chunks when cooled (left picture) in previously disclosed (micelles) method.

FIG. 4 is a schematic depicting certain steps for microbubble production after combining a first system comprising water-soluble surfactant and a second system comprising a water insoluble-surfactant. Example of oxygen filled MBs.

FIG. 5 demonstrates that SE61's (formed by combining Span 60 and TPGS) the zeta potential (¿ potential) is likely negative.

FIG. 6 show tables depicting size and (potential of SE61's MB population pre-freeze dry step for SE61's MBs prepared vial OM, MM, and CM.

FIG. 7 show tables depicting size and (potential of SE61's MB population post-freeze dry step SE61's MBs prepared vial OM, MM, and CM.

FIG. 8 shows a comparison of dose response curve post freeze drying for SE61 MBs prepared vial OM, MM, and CM.

FIG. 9 shows examples of possible additions for positively charged bubbles, the examples include cationic surfactants, cationic lipids, cationic cholesterols.

FIG. 10. shows N⁴-Cholesteryl-Spermine HCl Salt (GL67) structure, zeta potential, size distribution, and dose response for MBs prepared from GL67.

FIG. 11A shows the chemical structure of Rose Bengal (RB) sodium salt.

FIG. 11B shows a cartoon of proposed/putative SE61 microbubble structure prepared from Span 60 and TPGS.

FIG. 12 shows a schematic of sonodynamic therapy, wherein the MB are destroyed using low frequency ultrasound to release a therapeutic agent.

FIG. 13 shows a cartoon of putative CTAB-RB microbubble.

FIG. 14 shows a schematic of a process for making CTAB-RB microbubbles, according to the embodiments of the present invention.

FIG. 15 shows a set-up of in vitro acoustical testing of microbubbles to assess MB dose and time response.
bottom two images are taken under fluorescence. In the SE61-RB batch, there is no glowing indicating no RB attachment. In the CTAB-RB batch there is a glowing shell indicating successful RB attachment.

FIG. 16 is a table showing examples of cationic additives as well as their respective addition methods.

FIGS. 17A-17C size distributions of MBs with 5% (FIG. 17A), 15% (FIG. 17B), and 25% (FIG. 17C) cationic additive (post-sonication). Increasing the concentration of DDAB and DOTOP reduces the number of MBs created, with 15% and 25% DOTAP not producing sufficient MBs for testing.

FIGS. 18A-18C show that all cationic additives (5% (FIG. 18A), 15% (FIG. 18B), and 25% (FIG. 18C)) increased the zeta potential of SE61 (−40 mV pre-freeze dried, −30 mV post-freeze dried). Approximately 15% additive is needed to reach the target of 30 mV.

FIG. 19A-19C, all successful cationic modified SE61 MBs comprising 5% (FIG. 19A), 15% (FIG. 19B), and 25% (FIG. 19C) additives) produced an enhancement greater than 15 dB.

FIGS. 20A-20D are images of Unmodified SE61-RB brightfield (FIG. 20A) and fluorescence (FIG. 20B); 15% CTAB SE61-RB brightfield (FIG. OC), and fluorescence (FIG. 20D).

FIG. 21A-21B are images of in vivo ultrasound of a human head and neck tumor (arrows) (CAL27) in a nude mouse model, pre-injection (FIG. 21A) and post-injection (FIG. 21B).

FIG. 22 is a cartoon/putative structure of SE61 MB with examples of gases that can be filled within the MBS.

FIG. 23 is a schematic showing fabrication of SE61 (cream method).

FIG. 24 shows a dose response curves and sizes for MBs surface-modified using DMTAP at different concentrations.

FIG. 25 shows zeta potential for MBs surface-modified using 1,2-dimyristoyl-3-trimethylammonium-propane chloride (DMTAP) at different concentrations.

FIG. 26 is a graph showing DNA loading on MBs surface-modified using DMTAP at different concentrations

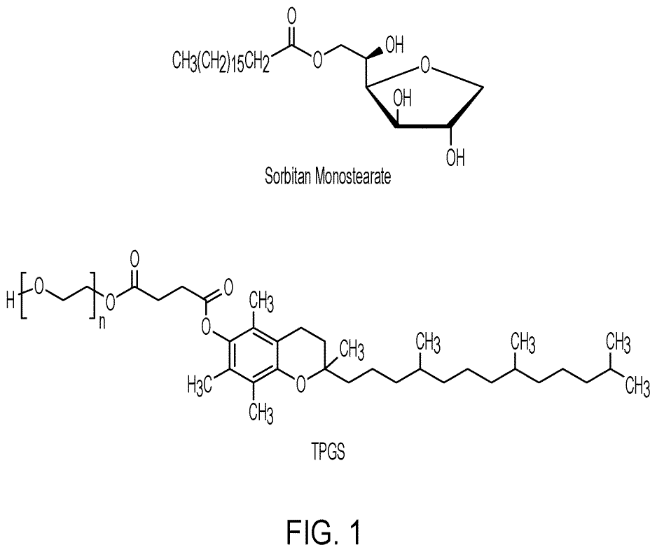

### DETAILED DESCRIPTION

In one aspect, the present invention is related to a process of making MBs comprised of at least two surfactant phases, one of which is water soluble and the other that is insoluble. The method allows for the creation of the two surfactant phases independently with the ability to incorporate additional species, such as drugs or charged molecules, in either phase at temperatures near room temperature to preserve the integrity of the additive. This method produces MBs with similar sizes distributions, surface charges, and acoustical properties as previous methods.

In another aspect, the present invention is related to incorporating a charged species into the MB shell. This charged species can be added to either the soluble or the insoluble phase of the preparation. This charged species can change the surface charge of the resulting MB and be used to facilitate the attachment of oppositely charged species. Previous methods had a surface charge of about −30 mV, while adding cationic molecules changed the charged surface to around +30 mV.

In another aspect, the present invention is related to attaching charged species to a surface modified MB, wherein the charged species is, for examples, Rose Bengal (RB) or nucleic acid to facilitate delivery using MBs and ultrasound to release drug at a target site. Since RB and nucleic acids are water soluble, typical encapsulation in the hydrophobic MB shell results in minimal loading. By adding cationic surfactants the MB becomes positively charged, allowing the negatively charged RB or nucleic acid to be attached. Attaching RB to a MB allows for drug and encapsulated gas e.g., O₂ delivery through Sonodynamic Therapy (SDT), which, uses ultrasound to initiate inertial cavitation. MB collapse releases drug and gas (O₂) from the core. Collapse triggers emission of photo-luminescence light which excites RB; and a further reaction with the released oxygen provokes biological damage. SDT is an improved alternative to chemotherapy. Chemotherapy is systemic, whereas SDT is a localized treatment and can target a specific tumor site.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Method

In one aspect, the invention provides a method for preparing microbubbles (MBs), wherein the method comprises the steps of providing a first system comprising at least one water-soluble surfactant (first surfactant); providing a second system comprising at least one water-insoluble surfactant (second surfactant), wherein providing the second system optionally comprises the step of autoclaving the second system and optionally cooling the resulting autoclaved system; and contacting the first system with the second system at the temperature below the melting point of the second surfactant to form a surfactant mixture comprising microbubbles.

In certain embodiments, the contacting is performed at about 100° C., 95° C., 90° C., 85° C., 80° C., 75° C., 70° C., 65° C., 60° C., 55° C., 50° C., 45° C., 40° C., 35° C., 30° C., 25° C., 20° C., 15° C., 10° C., 5° C., or 0° C. In certain embodiments, the contacting is performed at the room temperature.

In certain embodiments, the first system is aqueous. In certain embodiments, the first system comprises a micelle. In certain embodiments the concentration of the water-soluble/micelle-forming surfactant is above the critical micelle concentration (CMC).

The micelle-forming surfactant may include one or more micelle-forming surfactants such as diblock copolymers. The hydrophobic portions of such diblock copolymers may include one or more hydrophobic polymers, such as polyesters, polyanhydrides, polyglycolic acids, polybutrylactones, polyhydroxybutyrates, polylactic acids and polylacaprolactones. The hydrophobic portion of the copolymer may include one or more different hydrophobic polymers in random or block orientation.

Preferred hydrophilic portions of micelle forming copolymers that may be used in this invention have a molecular weight of about 750 or greater up to about 8000.

Non-limiting examples of the water-soluble surfactants include TPGS and/or suitable polyoxyethylene fatty acid esters such as TWEEN 20, TWEEN 40, TWEEN 60, TWEEN 65 and TWEEN 80, each of which are commercially available from ICI Americas, Inc. of Wilmington, Del. The TWEEN surfactants are each mixtures of various polyoxyethylene fatty acid esters in liquid form. For example, TWEEN 20 comprises polyoxyethylene (POE) esters of about 60 weight percent lauric acid (dodecanoic acid); about 18% myristic acid (tetradecanoic acid); about 7% caprylic acid (octanoic acid) and about 6% capric acid (decanoic acid). TWEEN 40 generally comprises POE esters of about 90% palmitic acid (hexadecanoic acid). TWEEN 60 generally comprises POE esters of about 49% stearic acid (octadecanoic acid) and about 44% palmitic acid. TWEEN 80 generally comprises POE esters of about 69% oleic acid (cis-9-octadecanoic acid); about 3% linoleic acid (linolic acid); about 3% linolenic acid (9,12,15-octadecatrienoic acid); about 1% stearic acid and about 1% palmitic acid. Additional examples include other water-soluble species that from micelles such as water-soluble diblock copolymers. Examples include those based on hydrophilic poly(ethylene glycol) (PEG).

The water-insoluble surfactant (second surfactant) is a dispersible surfactant, although one of ordinary skill in the art would understand that the second surfactant may be partially or fully soluble in a solvent. In certain embodiments, it is preferred that the second surfactant is non-ionic.

Non-limiting examples of the water-insoluble surfactants include sorbitan fatty acid esters, such as sorbitan monostearate, sorbitan monopalmitate and mixtures thereof. Preferred mixtures of sorbitan fatty esters include SPAN 40 and SPAN 60, each of which are dry powders commercially available from ICI Americas, Inc. SPAN 40 comprises sorbitan esters of about 93% palmitic acid; about 2.5% myristic acid and less than about 1% pentadecanoic acid. SPAN 60 comprises sorbitan esters of about 50% stearic acid, about 45% palmitic acid and about 2% myristic acid. Additional second components could be compounds such as (+) A tolopherol acid succinate (vitamin E), Brij 52, or Polyethylene-block-poly(ethylene glycol) MW 575.

In certain embodiments, the second system comprises a cream. In certain embodiments, the second system comprises an emulsion.

In certain embodiments, the method further comprises the step of agitating the surfactant mixture to form microbubbles. In certain embodiments, the method comprises sonicating the surfactant mixture to form the microbubbles. In certain embodiments, a homogenizer/homogenization is used to form the microbubbles.

In certain embodiments, the method further comprises purging the surfactant mixture with a purging gas.

In certain embodiments, the purging gas is a perfluorocarbon (PFC). In certain embodiments, the purging is performed with octafluropropane. In certain embodiments the purging gas is oxygen, sulfur hexafluoride (SF6), Perfluorobutane (C4F10) or other non toxic gas.

In certain embodiments, the method further comprises washing the mixture comprising microbubbles with PBS or DI water and separating the microbubbles from the mixture by gravity filtration. In certain embodiments, the method further comprises freeze-drying and capping the separated microbubbles under vacuum.

Generally, in certain embodiments, the frozen samples were lyophilized in vials with stoppers placed on the vials to the first groove, and placed on a previously chilled (−20° C.) shelf in a Virtis Benchtop freeze-dryer (Gardiner, NY). At the end of the cycle, prior to venting, a piston was lowered to seal the stoppers on the vials under vacuum.

In certain embodiments, the method further comprises filling the freeze-dried microbubbles with a filling gas. In certain embodiments, the method comprises reconstituting the freeze-dried bubbles with a water or PBS.

In certain embodiments, the filling gas is for example, oxygen, octafluoropropane xenon, nitrous oxide, nitrogen, C4F10 or sulfur hexafluoride, which is encapsulated by the MBs. In certain embodiments, the encapsulated gas is a therapeutic such as oxygen to facilitate treatment of hypoxia and to generate reactive oxygen species. In certain embodiments, the encapsulated gas is released upon insonation.

In certain embodiments, at least one of the first system and the second system independently comprises an additive.

In certain embodiments, at least one additive is selected from the group consisting of a drug, a dye, a polymer comprised of a nucleic acid and a charged molecules for surface modification.

In certain embodiments, the method allows for the separate manipulation of the two surfactants prior to the final mixture to allow for the addition of additives in either the micelle (first system comprising water-soluble surfactant) or the cream (second system comprising water-insoluble surfactant), thus avoiding deleterious effects of heat, drug interaction and/or surfactant precipitation.

Additives can include drugs, dyes, and/or charged molecules for surface modification. Charged molecules can be cationic, anionic, or zwitterionic: surfactants, lipids, sterols such as cholesterols e.g., negatively charged cholesterol sulfate (cholesterol-SO₄) and positively charged 3 β-[N-(dimethylaminoethane) carbamoyl]-cholesterol (DC-cholesterol), zwitterionic surfactants such as CHAPS (3-[(3-Cholamidopropyl)dimethylammonio]-1-propanesulfonate).

In certain embodiments, addition of additives can change the surface charge of the microbubble. In certain embodiments, additives create MBs having positive charge.

In certain embodiments, the examples of additives that modify the surface of the MBs include CTAB, DSTAP, DMTAP, DDAB, TAP 18:0, GL67, and DOTOP.

In other embodiments, the charged MB can be further loaded with oppositely charged molecules, such as but not limited to DNA, RNA, and/or Rose Bangel. In certain embodiments, the charged MB is subjected to a wash step in which species of the opposite charge are attracted loaded on to the shell by ionic interaction. For example, the MBs with positive zeta potential can further be loaded with negatively charged molecules such as, for example, therapeutic agents including Rose Bengal and DNA.

In certain embodiments, MBs can be loaded with a charged species such as genetic material in cases such but not limited to DNA, RNA, siRNA, shRNA, and/or CRISPR. In certain embodiments, the charged species is a sonosensitizer.

In certain embodiments, the charged species is a positively charged protein or peptide, for example (but not limited to) to allow for delivery across the high negative fixed charge density (FDC) of the anionic proteoglycans in various tissues (including but not limited to cartilage, meniscus and ligaments of the knee, ankle, shoulder and hip joints etc., intervertebral discs. (IVDs) in the spine, mucosal membrane of the gastrointestinal (GI) tract and the vitreous humor of the eye, and skin).

In certain embodiments, opsonization upon intravenous injection is minimized by a positive surface charge.

In certain embodiments, the charged species is effective against multi drug resistance.

In certain embodiments, the MBs are acoustically-sensitive.

In certain embodiments, in step the size of the separated microbubbles is less than about 10, 9, 8, 7, 6, 5, 4, 3, 2, 1 μm.

### Kit

In another aspect, the invention provides a kit comprising surfactants and reagents for making the MBs as described elsewhere herein and instruction material for making the MBs.

### EXPERIMENTAL EXAMPLES

The invention is further described in detail by reference to the following experimental examples. These examples are provided for purposes of illustration only and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods.

Bubble Counting and Sizing: AccuSizer (A7000 Single Particle Optical Sizing, Entegris). Three samples taken from each vial and bubble size and diameter were averaged.

Charge Analysis: ZetaSizer (Zetasizer Nano ZS, Malvern Instruments)—Three samples taken with three readings each per vial then averaged to find particle charge.

Acoustical Testing: Pulsed, A-mode ultrasound system with a 5 MHz transducer used to mimic in-vivo conditions

Dose Response: Reflected signal (dB) as a function of increasing dose. Minimum 15 dB needed for in vivo contrast imaging

Time Response: Reflected signal from one dose (180 μl/L) over 10 min. Normalize to determine half-life

Plate Reader: (Synergy H1, BioTek microplate reader); Standard curve developed using serial dilution of RB stock solution at concentration of 20 μg/mL. Costar 96 Black opaque, Excitation: 525, Emission: 570, Gain: 0, Xenon Flash. Tested bubbles reconstituted with ethanol and no gas

Microscope Imaging: Fluorescent filter TRITC, images taken at an exposure of 39 ms. Photos were taken in brightfield before transferring to the fluorescent filter to confirm bubble presence.

Acoustic properties of the MBs were assessed in vitro in a tank setup consisting of a pulsed A-mode ultrasound system fitted with an exchangeable Olympus (Waltham, MA) 5 MHz transducer with a 12.7 mm diameter and a focal length of 49.3 mm. Acoustic pressure amplitudes were generated using a Panametrics pulser/receiver (model 5072 PR) using a pulse repetition frequency (PRF) of 100 Hz generating peak positive and negative pressures of 0.69 and 0.45 MPa, respectively. Received signals were amplified 40 dB and read using a digital oscilloscope (LeCroy 9350A, LeCroy Corp., Chestnut Ridge, NY), and the data were processed using Lab View (National Instruments, Austin, TX). The transducer was focused through an acoustic window of a custom-made sample vessel submerged in a deionized water bath (37° C.), with the contents continuously stirred during testing. Cumulative dose response curves, that is signal returned to the transducer as a function of microbubble dose, were constructed by pipetting increments of MBs into the sample chamber containing 50 mL of phosphate buffered saline at 37° C. while measuring the acoustic response. To examine the stability of the MBs while being exposed continuously to an ultrasound beam (time response or stability curve) a dose on the rise of the dose response curve was insonated over a 10 minute period using the same acoustic parameters used for the dose response studies. Readings were taken every minute, starting at t=0 (time immediately post injection), for a total of 11 readings, with data normalized by the initial dB value to allow for comparison.

### Example 1: Synthesis of Surfactant-Stabilized Microbubbles (SE61) Using the Cream Method

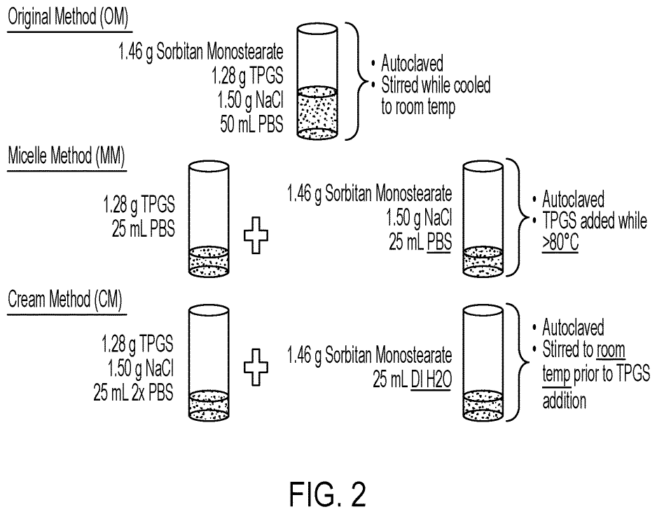

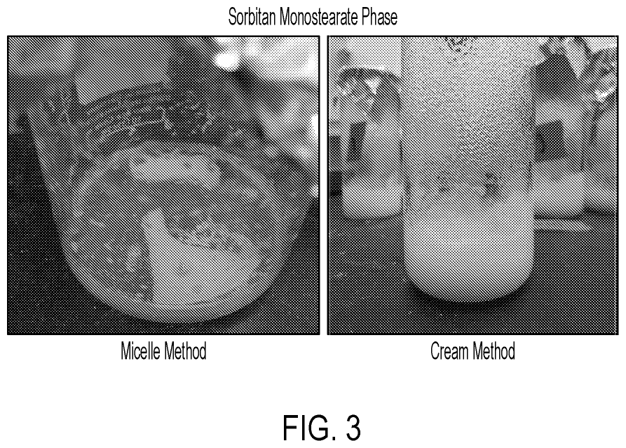

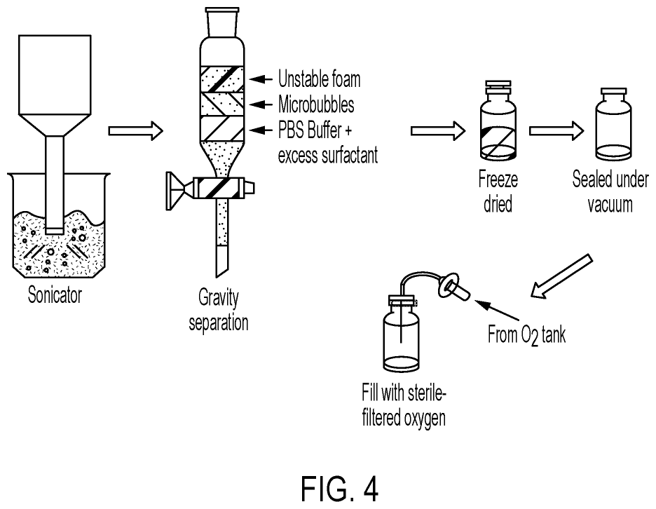

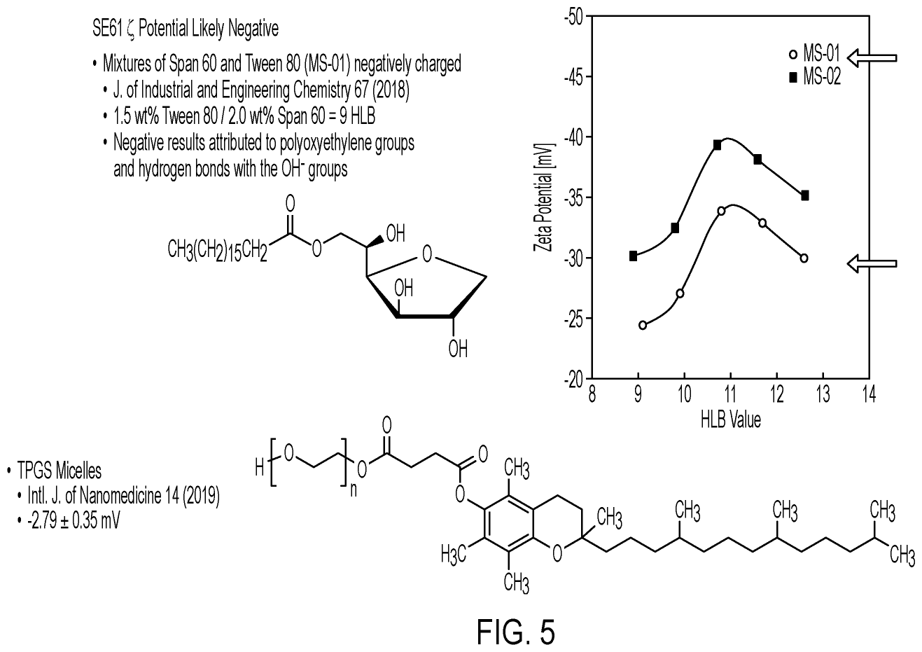

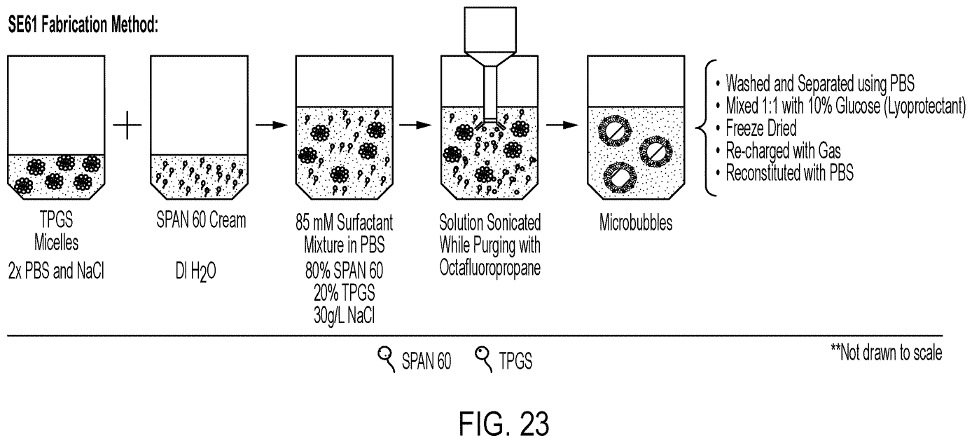

Mixtures of 1.5 g NaCl, 1.464 g sorbitan monostearate (Span 60) and 1.29 g TPGS in 50 mL phosphate buffered saline (PBS) to produce SE61 MBs were made using three different methods as shown in FIGS. 2 and 23. For the Original Method (OM), all components were combined into a 250 ml beaker and autoclaved for about 35 minutes, then cooled to room temperature while stirred. For the Micelle Method (MM), TPGS was combined with 25 mL of PBS to form micelles, and separately, Span 60 and NaCl were combined with 25 mL of PBS and autoclaved. After autoclaving but before cooling below 80° C., the Span 60 mixture was added to the TPGS micelle solution, and the final mixture allowed to cool to room temperature. For the Cream Method (CM), TPGS and NaCl was added to 25 mL of 2×PBS to form micelles, and separately, Span 60 was added to 25 mL of DI water and autoclaved. After autoclaving, the Span 60 mixture was allowed to cool to room temperature while stirring. Once cooled to room temperature and a cream had formed (FIG. 3), the TPGS solution was added to the Span 60 cream for the final mixture.

These mixtures were each purged with PFC gas (octafluoropropane) and sonicated for 3 minutes at 100-120 W to create a microbubble solution. The solution was further added to a gravity separation funnel and washed three times with 50 mL deionized (DI) water. Between washes, the bottom layer was drained and discarded. After the final wash and discarding the bottom layer, the middle, bubble layer was collected, and combined with 10% glucose in a 1:1 volume ratio. MBs were pipetted in 4 mL aliquots into 10 mL vials, and frozen using a −20° C. bath. The MBs were then lyophilized and vials were sealed under vacuum using a rubber stopper. Before further testing was conducted on these microbubbles, vials were filled with PFC gas and the microbubbles were reconstituted with 0.5×PBS.

### Results

Results were compared between three batches of bubbles for each method.

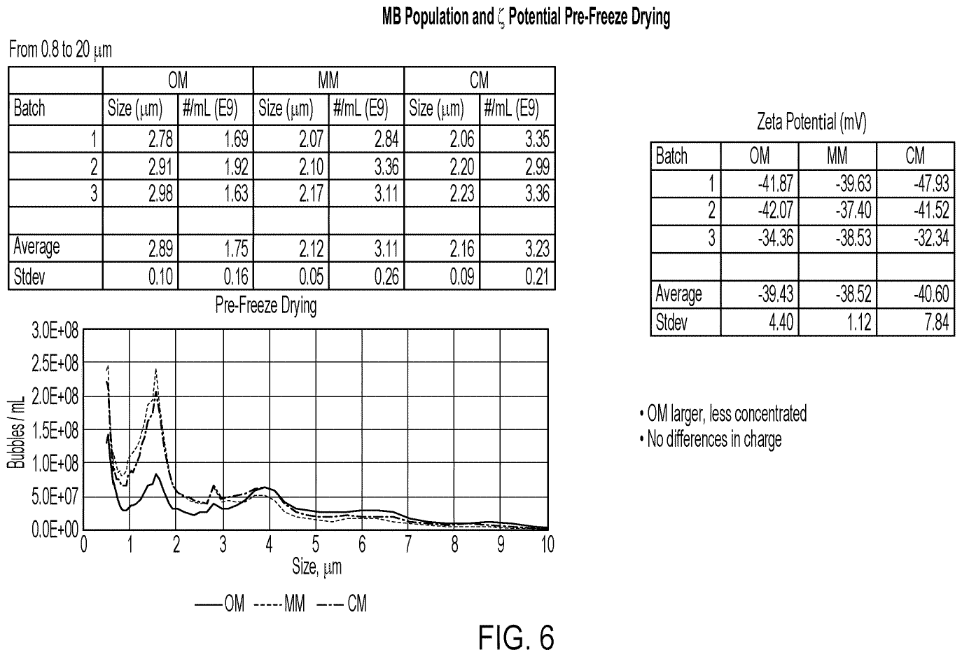

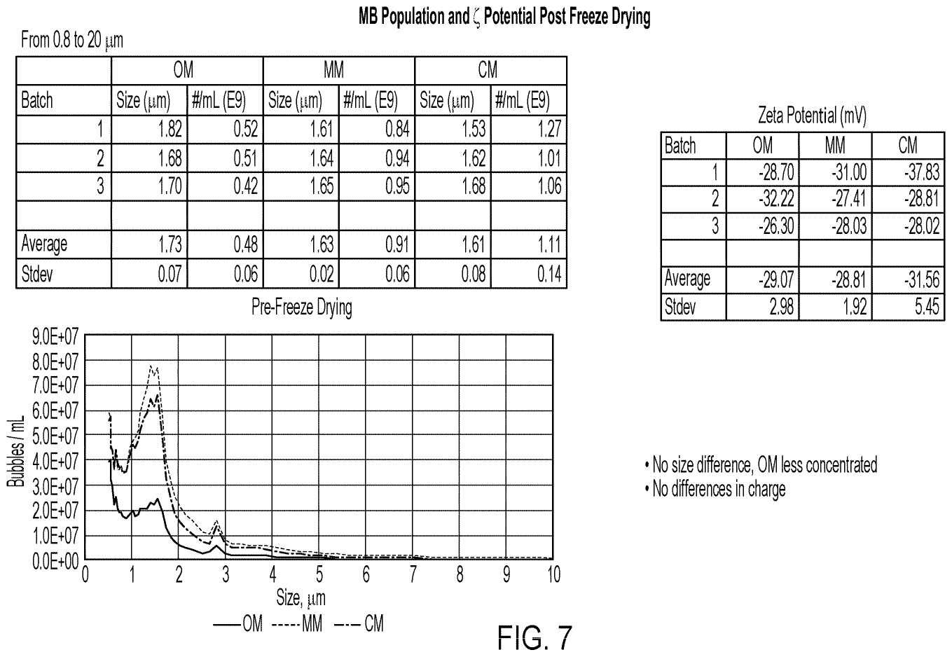

Bubble Counting and Sizing: MB populations were determined pre and post freeze drying for each method. Pre-freeze drying (FIG. 6), the OM had a larger average size (2.89 μm) and lower concentration (1.75 E9 bubbles/mL) than the MM (2.12 μm and 3.11 E9 bubbles/mL) and CM method (2.16 μm and 3.23 E9 bubbles/mL). Post-freeze drying (FIG. 7) there was no size difference, but OM again had a lower concentration than MM and CM.

Charge analysis: All methods produced MBs with a negatively charged surface, with zeta potentials near −40 pre-freeze drying (FIG. 6) and near −30 post freeze drying (FIG. 7).

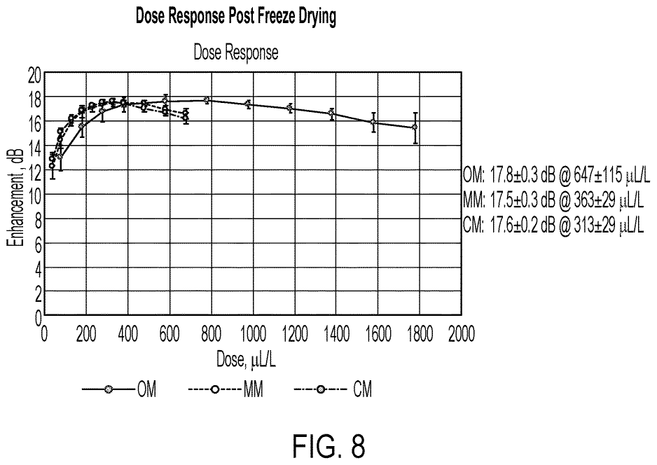

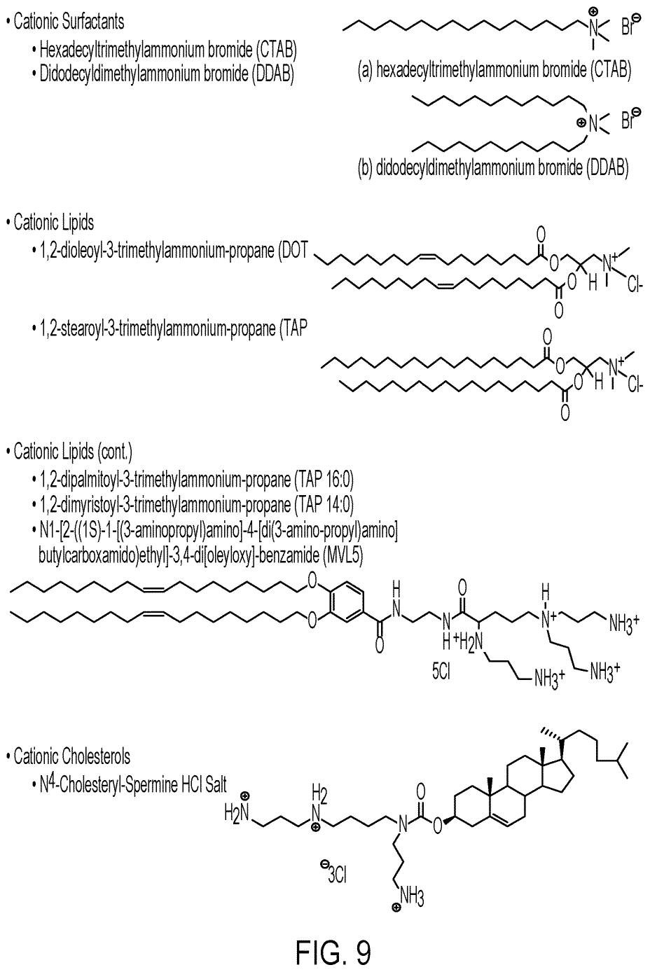

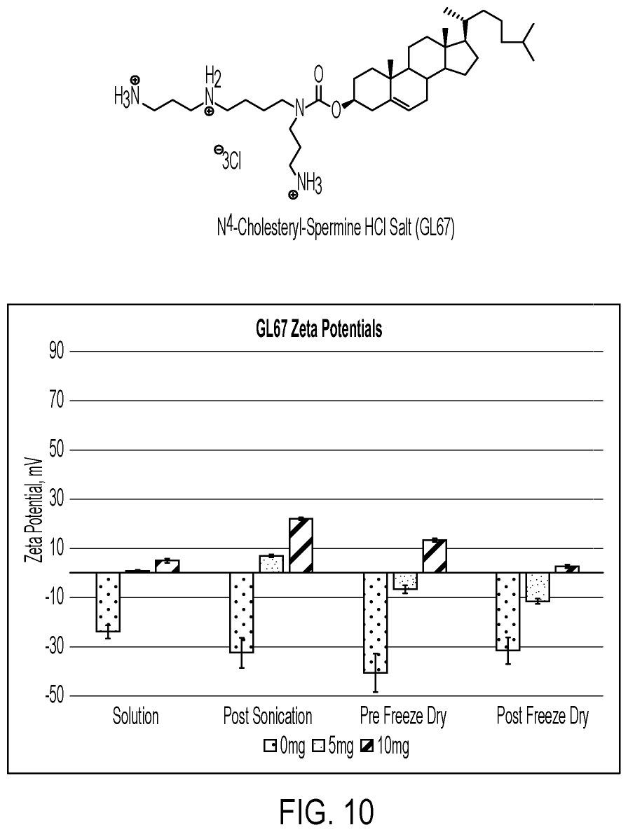

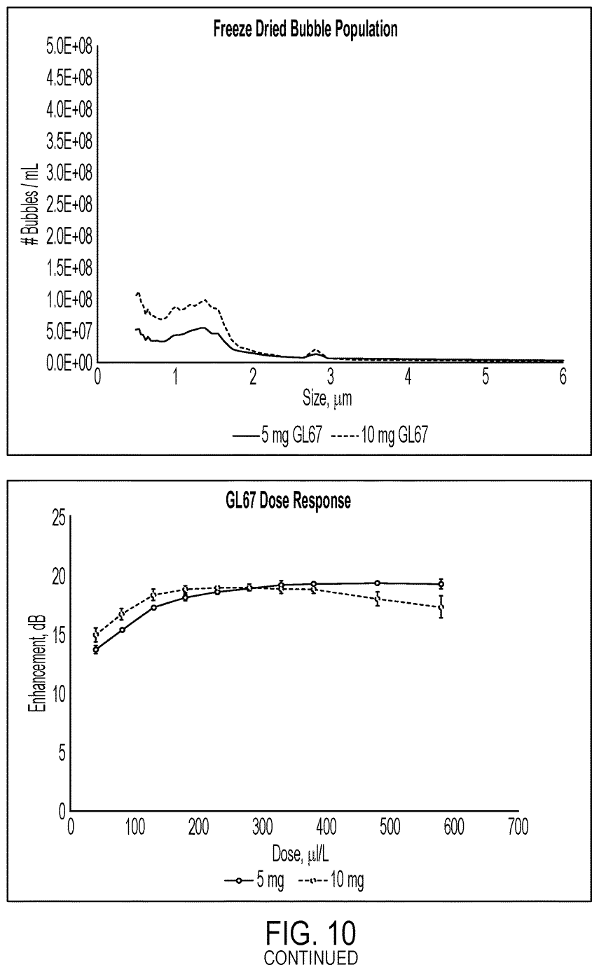

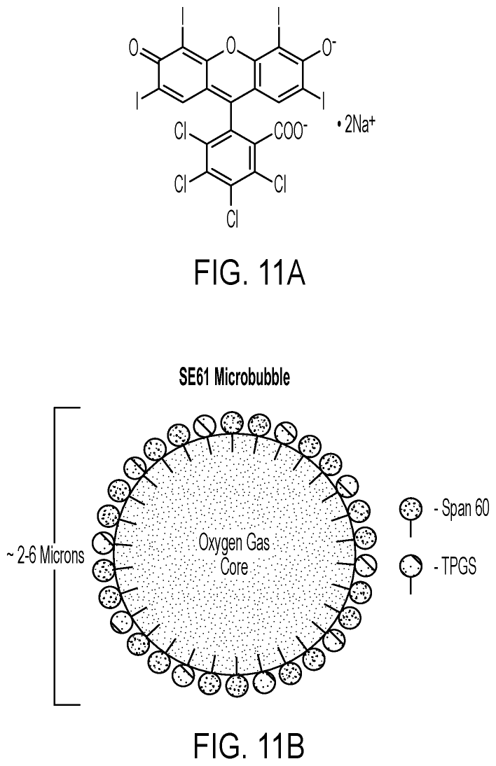

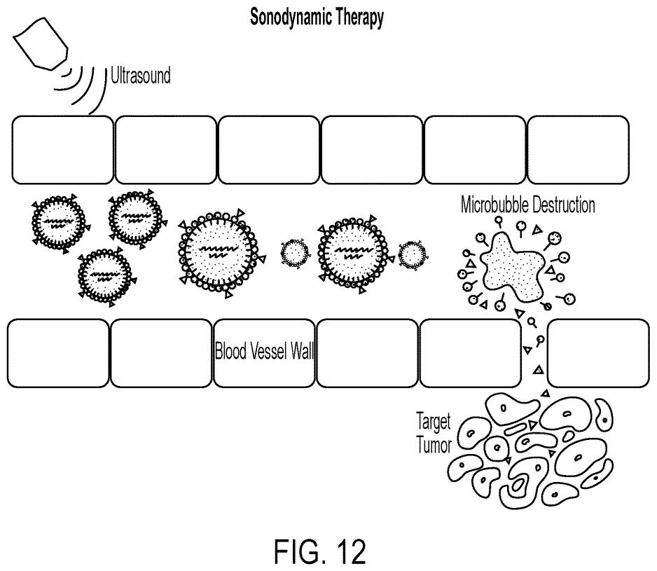

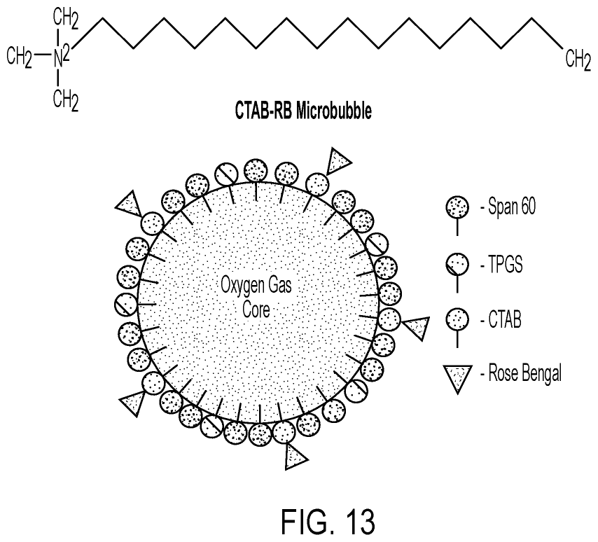

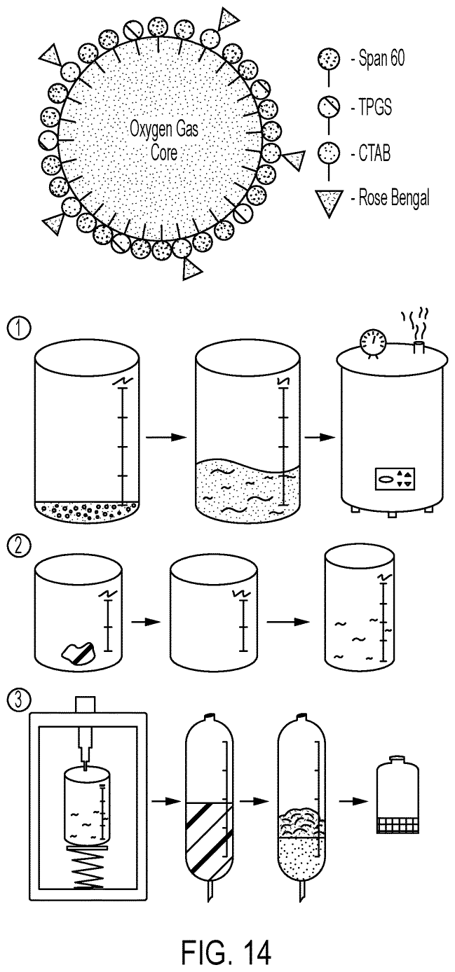

Acoustical Testing: The Cream Method (CM) does not negatively affect the acoustic properties of the MB compared to the Micelle Method (MM) and the original Method (OM) as demonstrated by the similar maximum enhancement on the dose response curve (FIG. 8). Both the CM and the MM produced the maximum enhancement at a lower dose due to the increased bubble concentration.

These results show that the CM produces SE61 MBs with the same size population, surface charge, and acoustical properties as the MM, while allowing both the TPGS and the SPAN components to cool to room temperature before mixing. The CM also allows for additions to the Span post autoclaving.

### Example 2: Modifying SE61 by Adding Cationic Species

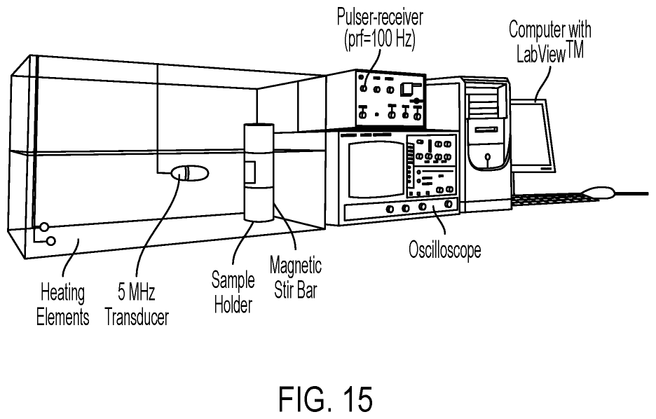

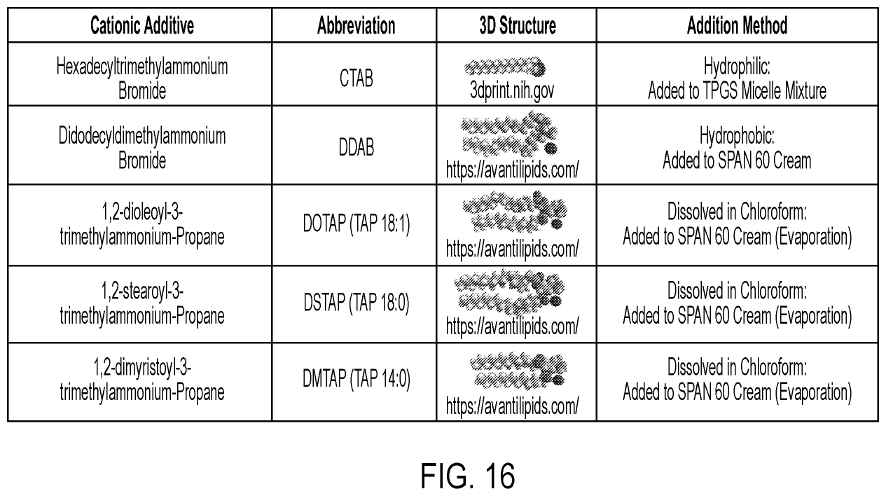

SE61 MBs were modified by adding cationic species to either the water soluble or water insoluble portion of the cream method (CM). While maintaining an overall 85 mM surfactant mixture and relative 4:1 ratio of SPAN 60 to TPGS, three molar ratios were tested: 5% Cationic Additive, 76% SPAN 60, 19% TPGS; 15% Cationic Additive, 68% SPAN 60, 17% TPGS; 25% Cationic Additive, 60% SPAN 60, 15% TPGS. Cationic additive and their respective addition methods are shown in the table depicted in FIG. 16.

Microbubble Testing: analyses post sonication, pre-freeze drying, and post-freeze drying were performed. The testing included testing bubble populations (using AccuSizer A7000, Entegris Inc.) zeta analysis (using Zetasizer Nano ZS, Malvern Inst.), and testing dose response (Freeze dried only), which included measuring reflected signal (dB) as function of increasing dose. Minimum 15 dB needed for in vivo contrast imaging.

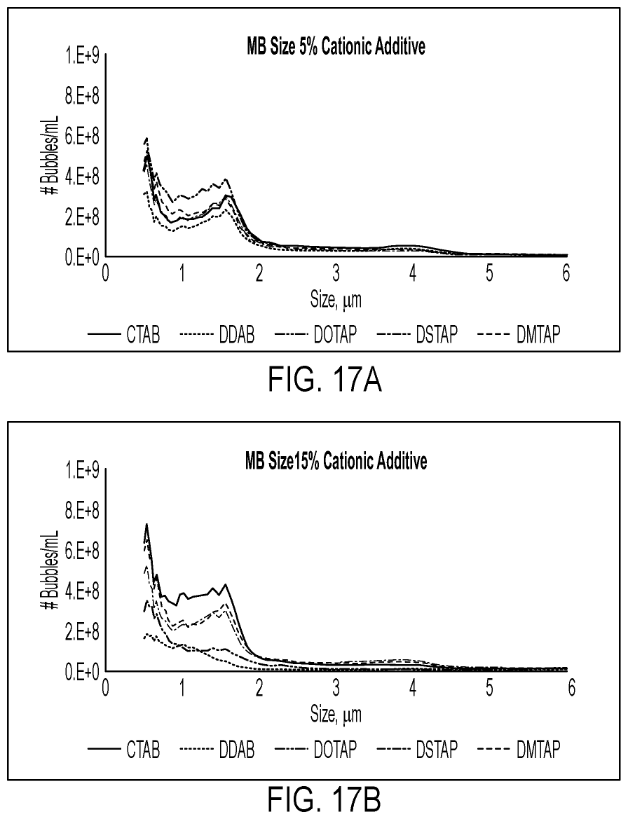

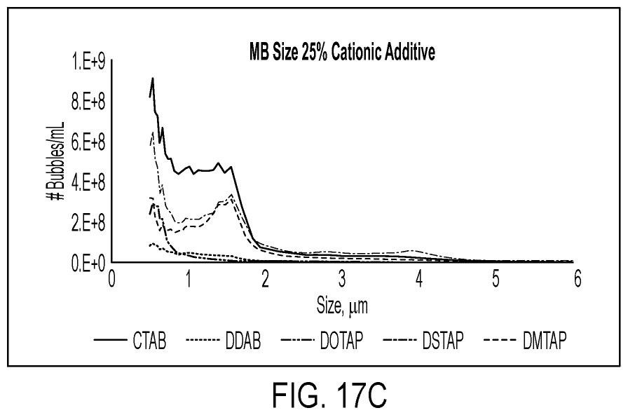

As shown in FIGS. 17A-17C, size distributions (post-sonication) show MBs created with all 5% cationic additives. Increasing the concentration of DDAB and DOTOP reduces the number of MBs created, with 15% and 25% DOTAP not producing sufficient MBs for testing.

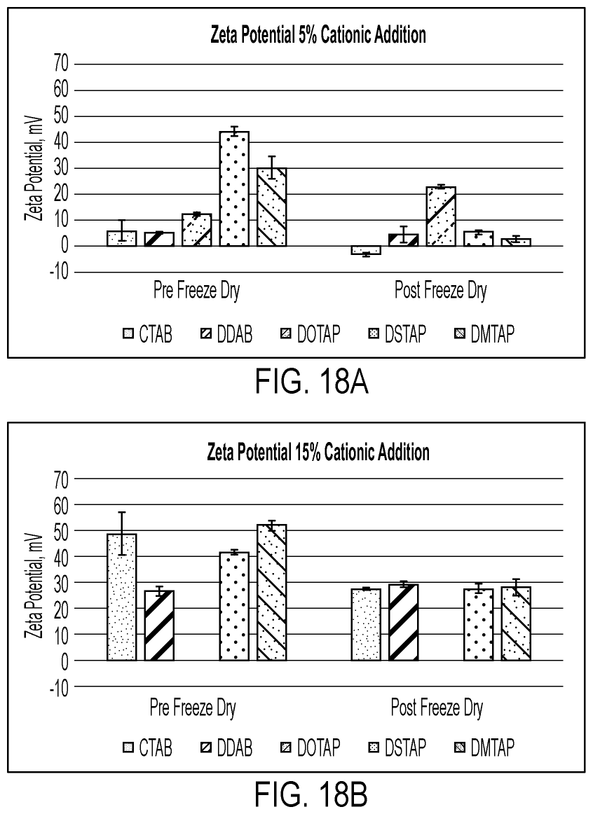

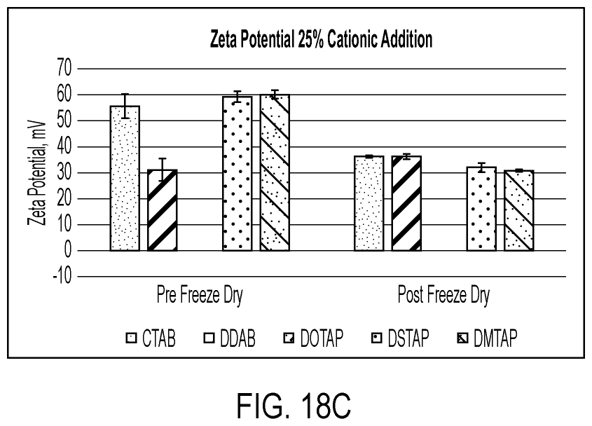

As shown in FIGS. 18A-18C, all cationic additives increased the zeta potential of SE61 (−40 mV pre-freeze dried, −30 mV post-freeze dried). Approximately 15% additive is needed to reach the target of 30 mV.

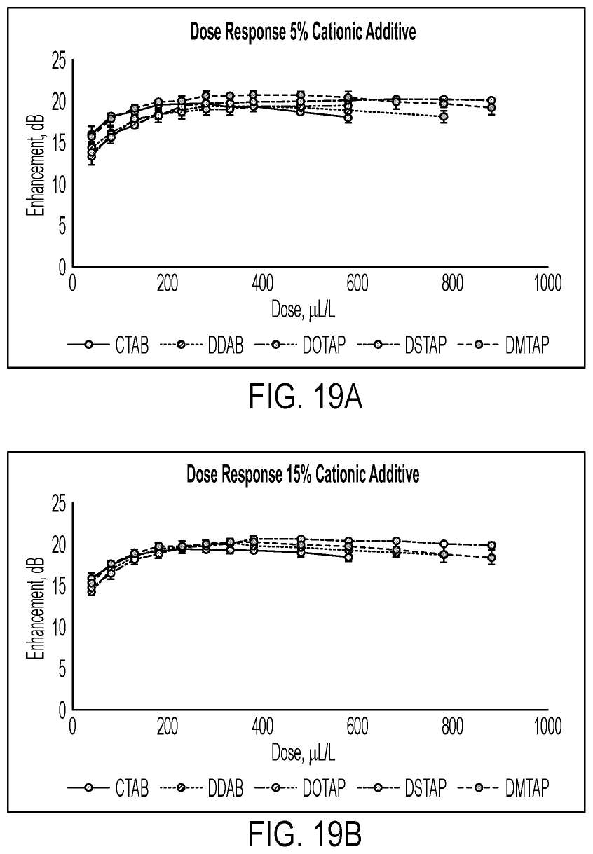

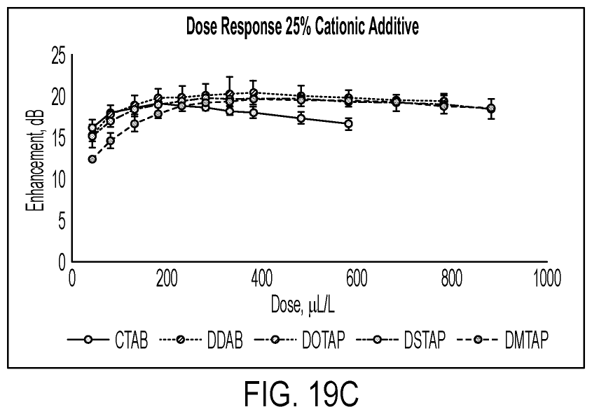

As shown in FIG. 19A-19C, all successful cationic modified SE61 MBs produced an enhancement greater than 15 dB.

These experiments demonstrated that addition of cationic molecules such as CTAB, DSTAP and DMTAP can create positively charged SE61 MBs.

### Example 3: Using 15% CTAB SE61 for Loading Negatively Charged Therapeutics

SE61 MBs were modified by adding CTAB to the water soluble portion of the cream method (CM) using a molar ratio of 15% CTAB, 68% SPAN 60, 17% TPGS.

Double stranded DNA used to model siRNA had a sequence of (5′-AATGAGCCCTTGCATCTAAGAA-3′) (SEQ ID No: 1). The process of DNA loading onto MB after wash and collection included 1 part 250 mM dsDNA, 1 part 20% w/v glucose (cryoprotectant), and 2 parts collected microbubbles. The MBs were washed with deionized water allowing dsDNA-loaded MB to separate due to gravity. Washes with unattached dsDNA were also collected and tested, (Quant-iT PicoGreen dsDNA assay, invitrogen). Detected amount of dsDNA was subtracted from initial amount. 373 mg/mL DNA/mL of MB were loaded (63.5% loading). No loading detected with unmodified SE61. These results indicate that nucleic acids can be loaded onto the modified MB shell.

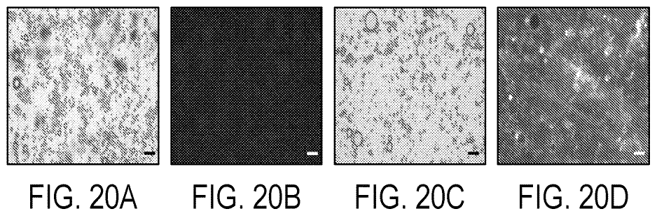

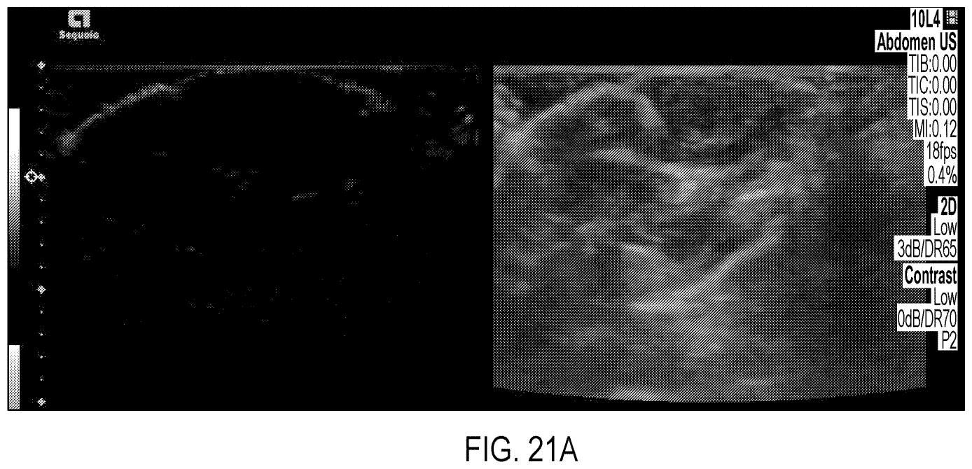

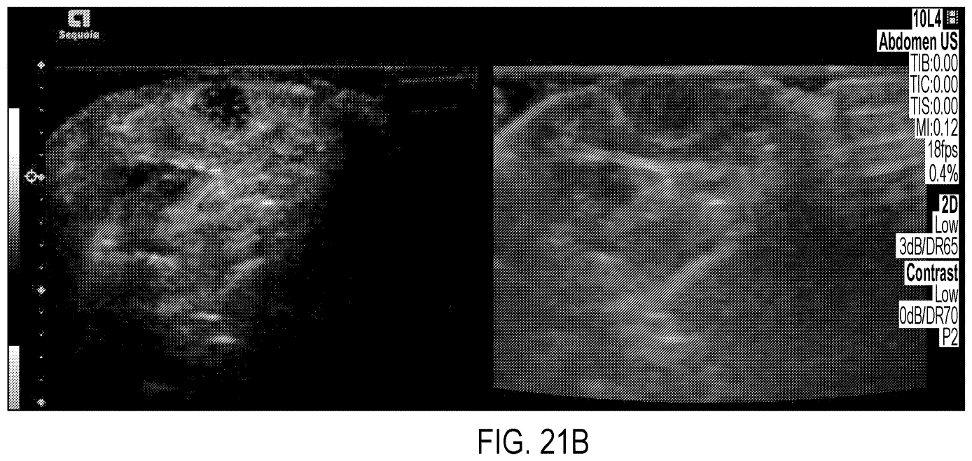

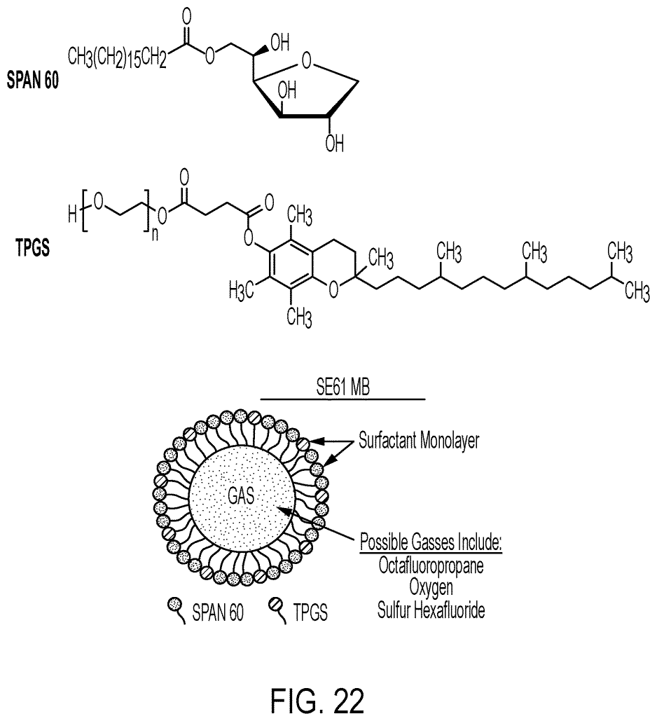

For fabricating RB loaded MBs, 100 mg of RB was added to approximately 120 mL of MBs after the first wash. RB Loading was detected by absorption at 560 nm (in ethanol) using Synergy H1 plate reader unmodified SE61 contained 1.5 μg RB/mL MB whereas as 15% CTAB SE61 contained 23.5 μg RB/mL MB. Optical Imaging was performed using IX71 Olympus fluorescent microscope (TRITC filter). Results for Unmodified SE61-RB and 15% CTAB SE61-RB brightfield and fluorescence are shown in FIGS. 20A-20D. In vivo Ultrasound Imaging was performed via continuous imaging in B-mode and contrast mode using a 10L4 transducer and an Acuson Sequoia, Siemens Healthineers. In vivo ultrasound images of a human head and neck tumor (arrows) (CAL27) in a nude mouse model, pre-injection and post-injection are shown in FIGS. 21B and 21B, respectively. The results of this experiment indicated successful loading of RB onto a MB made of surfactants TPGS, Span 60, and CTAB with an oxygen filled gas core. These results were compared to a SE61 MB that underwent the same RB wash process as the CTAB RB. In the SE61 MB no RB was attached confirmed by both the plate reader data and a fluorescent microscope. These results demonstrate excellent potential for the production of RB-MB for use with sonodynamic therapy (SDT).

### Example 4: Characterization of MBs with Various Amounts of DMTAP (TAP 14)

SE61 MBs were modified by adding DMTAP to the water insoluble soluble portion of the cream method (CM). While maintaining an overall 85 mM surfactant mixture and relative 4:1 ratio of SPAN 60 to TPGS, MBs with increasing amount of DMTAP (TAP 14) (0, 10, 15, 20, 25%) were prepared. MBs were loaded with double stranded DNA (dsDNA), wherein 320 μL collected microbubbles were loaded with 160 μL dSDNA (250 μM) in the presence of 160 μL 20% glucose cryoprotectant.

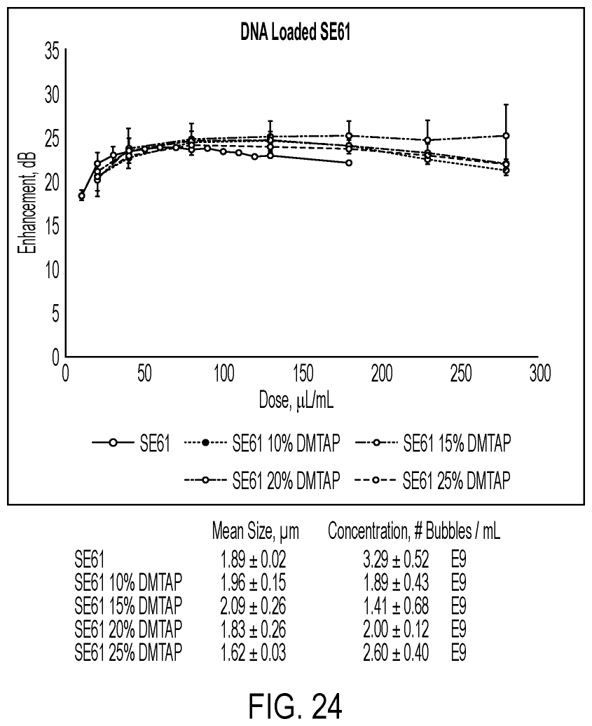

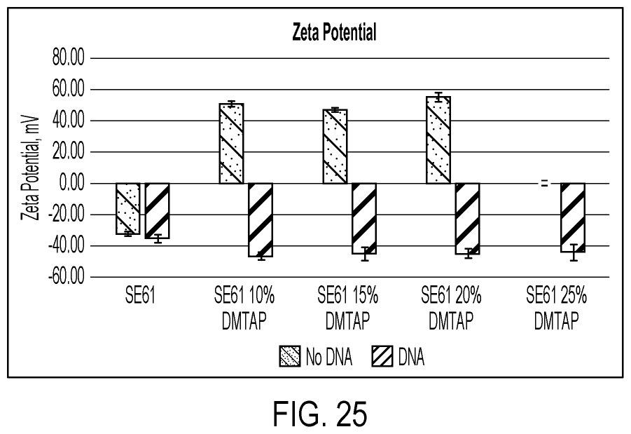

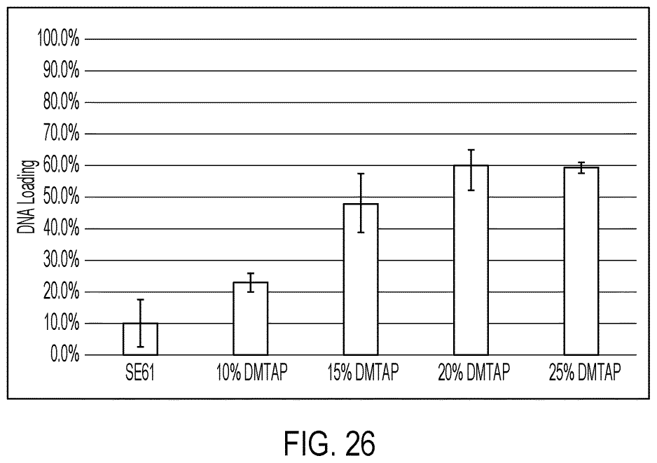

MBs were analyzed and the results related to dose response curves, size, Zeta Potential, and DNA loading are shown in FIGS. 24-26. These results indicate that a ratio of 20% DMTAP is optimal for loading nucleic acid.

### Enumerated Embodiments

The following exemplary embodiments are provided, the numbering of which is not to be construed as designating levels.

Embodiment 1 comprises a method for preparing microbubbles, wherein the method comprises:
i. providing a first system comprising at least one water-soluble surfactant;
ii. providing a second system comprising at least one water-insoluble surfactant, wherein providing the second system optionally comprises the step of autoclaving the second system and optionally cooling the resulting autoclaved system; and
iii. contacting the first system with the second system at the temperature below the melting point of the second surfactant, so as to produce a mixture comprising microbubbles.

Embodiment 2 provides the method of embodiment 1, wherein the first system is aqueous.

Embodiment 3 provides the method of embodiments 1-2, wherein the first system comprises a micelle.

Embodiment 4 provides the method of embodiments 1-3, wherein the second system comprises a cream.

Embodiment 5 provides the method of embodiments 1-4, wherein the method further comprises purging the surfactant mixture of step (iii) with a purging gas.

Embodiment 6 provides the method of embodiments 1-5, wherein the purging gas is a perfluorocarbon (PFC).

Embodiment 7 provides the method of embodiments 1-6, wherein the method further comprises washing the mixture comprising microbubbles with DI water and separating the microbubbles from the mixture by gravity filtration.

Embodiment 8 provides the method of embodiments 1-7, wherein the method further comprises freeze-drying and capping the separated microbubbles under vacuum.

Embodiment 9 provides the method of embodiments 1-8, the method further comprises filling the freeze-dried microbubbles with a filling gas.

Embodiment 10 provides the method of embodiments 1-9, wherein the filling gas is oxygen, octafluoropropane, and sulfur hexafluoride.

Embodiment 11 provides the method of embodiments 1-10, wherein the at least one water soluble surfactant is selected from the group consisting of tocopheryl polyethylene glycol succinate (TPGS), cetyltrimethylammonium Bromide (CTAB), and mixtures thereof.

Embodiment 12 provides the method of embodiments 1-11, wherein the at least one water-insoluble surfactant is selected from the group consisting of sorbitan monostearate, sorbitan fatty acid esters, sorbitan monopalmitate, (+) A tolopherol acid succinate (vitamin E) and mixtures thereof.

Embodiment 13 provides the method of embodiments 1-12, wherein the contacting is performed at room temperature.

Embodiment 14 provides the method of embodiments 1-13, wherein at least one of the first system and the second system independently comprises an additive.

Embodiment 15 provides the method of embodiments 1-14, wherein the at least one additive is selected from the group consisting of a drug, a dye, and a charged molecule for surface modification of the microbubbles.

Embodiment 16 provides the method of embodiments 1-15, wherein the charged molecule alters the charge (zeta potential) of the microbubbles.

Embodiment 17 provides the method of embodiments 1-16, wherein the microbubbles with altered zeta potential are further loaded with oppositely charged molecules.

Embodiment 18 provides the method of embodiments 1-17, wherein the microbubbles are acoustically-sensitive.

Embodiment 19 provides the method of embodiments 1-18, wherein the size of the separated microbubbles is below 10 μm in diameter.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations

What is claimed is:

1. A method for preparing microbubbles, wherein the method comprises:
i. providing a first system comprising at least one water-soluble surfactant;
ii. providing a second system comprising at least one water-insoluble surfactant, wherein providing the second system optionally comprises the step of autoclaving the second system and optionally cooling the resulting autoclaved system; and
iii. contacting the first system with the second system at the temperature below the melting point of the second surfactant, so as to produce a mixture comprising microbubbles.

2. The method of claim 1, wherein the first system is aqueous.

3. The method of claim 1, wherein the first system comprises a micelle.

4. The method of claim 1, wherein the second system comprises a cream.

5. The method of claim 1, wherein the method further comprises purging the surfactant mixture of step (iii) with a purging gas.

6. The method of claim 5, wherein the purging gas is a perfluorocarbon (PFC).

7. The method of claim 6, wherein the method further comprises washing the mixture comprising microbubbles with DI water and separating the microbubbles from the mixture by gravity filtration.

8. The method of claim 7, wherein the method further comprises freeze-drying and capping the separated microbubbles under vacuum.

9. The method of claim 8, the method further comprises filling the freeze-dried microbubbles with a filling gas.

10. The method of claim 9, wherein the filling gas is oxygen, octafluoropropane, and sulfur hexafluoride.

11. The method of claim 1, wherein the at least one water soluble surfactant is selected from the group consisting of tocopheryl polyethylene glycol succinate (TPGS), cetyltrimethylammonium Bromide (CTAB), and mixtures thereof.

12. The method of claim 1, wherein the at least one water-insoluble surfactant is selected from the group consisting of sorbitan monostearate, sorbitan fatty acid esters, sorbitan monopalmitate, (+) A tolopherol acid succinate (vitamin E) and mixtures thereof.

13. The method of claim 1, wherein the contacting is performed at room temperature.

14. The method of claim 1, wherein at least one of the first system and the second system independently comprises an additive.

15. The method of claim 14, wherein the at least one additive is selected from the group consisting of a drug, a dye, and a charged molecule for surface modification of the microbubbles.

16. The method of claim 15, wherein the charged molecule alters the surface-charge of the microbubbles.

17. The method of claim 16, wherein the microbubbles with altered surface-charge are further loaded with an oppositely charged molecule.

18. The method of claim 1, wherein the microbubbles are acoustically-sensitive.

19. The method of claim 1, wherein the size of the separated microbubbles is below 10 μm in diameter.
